(19) United States
(12) Patent Application Publication
(10) Pub. No.: US 2009/0048527 A1
(43) Pub. Date: Feb. 19, 2009

(54) ASSESSMENT OF PRELOAD DEPENDENCE AND FLUID RESPONSIVENESS

(75) Inventors: Feras Hatib, Irvine, CA (US); Lina Derderian, Trabuco Canyon, CA (US); Luchy Roteliuk, Lake Forest, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/190,188

(22) Filed: Aug. 12, 2008

Related U.S. Application Data
(60) Provisional application No. 60/955,588, filed on Aug. 13, 2007.

(51) Int. Cl. A61B 5/02

(52) U.S. Cl. 600/508

(57) ABSTRACT

Methods for determining a cardiovascular parameter reflecting fluid or volume changes and for detecting arrhythmia are disclosed. These methods involve receiving a waveform dataset corresponding to an arterial blood pressure, pulseox, Doppler ultrasound or bioimpedance signal and analyzing the waveform to detect premature ventrical or atrial contractions. Premature ventrical or atrial contractions are detected, for example, by comparing parameters of individual cardiac cycles to the parameters of other or average cardiac cycles. If any premature ventrical or atrial contractions are present, they are removed from the waveform dataset. Once any the premature ventrical or atrial contractions are removed, a cardiovascular parameter reflecting fluid or volume changes using the modified waveform dataset is calculated. In the method for determining arrhythmia, if the number of premature ventrical or atrial contractions exceeds a predetermined arrhythmia threshold, a user such as a medical professional is notified.

### CLAIM OF PRIORITY UNDER 35 U.S.C. §119

The present Application for patent claims priority to Provisional Application No. 60/955,588 filed Aug. 13, 2007, and assigned to the assignee hereof and hereby expressly incorporated by reference herein.

### BACKGROUND

Indicators such as stroke volume (SV), cardiac output (CO), end-diastolic volume, ejection fraction, stroke volume variation (SVV), pulse pressure variation (PPV), and systolic pressure variations (SPV), among others, are important not only for diagnosis of disease, but also for “real-time” monitoring of preload dependence, fluid responsiveness, or volume responsiveness condition of both human and animal subjects. Few hospitals are therefore without some form of equipment to monitor one or more of these cardiac parameters. Many techniques, including invasive techniques, non-invasive techniques, and combinations thereof, are in use and even more have been proposed in the literature.

Many of the techniques used to measure SV can be adapted to provide an estimate of CO as well, because CO is generally defined as SV times the heart rate (HR), which is usually available to monitoring equipment. Conversely, most devices that estimate CO also estimate SV in their calculations. One way to estimate SVV is simply to collect multiple SV values and calculate the differences from measurement interval to measurement interval.

One way to measure SV or CO is to mount a flow-measuring device on a catheter, and position the device in or near the subjects heart. Some such devices inject either a bolus of material or energy (usually heat) at an upstream position, such as in the right atrium, and determine flow based on the characteristics of the injected material or energy at a downstream position, such as in the pulmonary artery. Patents that disclose implementations of such invasive techniques (in particular, thermodilution) include: U.S. Pat. No. 4,236,527 (Newbower et al., 2 Dec. 1980); U.S. Pat. No. 4,507,974 (Yelderman, 2 Apr. 1985); U.S. Pat. No. 5,146,414 (McKown, et al., 8 Sep. 1992); and U.S. Pat. No. 5,687,733 (McKown, et al., 18 Nov. 1997). Other invasive devices are based on the known Fick technique, according to which CO is calculated as a function of oxygenation of arterial and mixed venous blood.

Invasive techniques have obvious disadvantages, especially when the subjects in need of such monitoring are already in the hospital due to a serious condition. Invasive methods also have less obvious disadvantages, for example, some techniques such as thermodilution rely on assumptions, such as uniform dispersion of the injected heat, that affect the accuracy of the measurements. Moreover, the introduction of an instrument into the blood flow may affect the value that the instrument measures.

Doppler techniques, using invasive as well as non-invasive transducers, have also been used to obtain flow rate data that can then be used to calculate SV and CO. However, these systems are typically expensive, and their accuracy depends on precise knowledge of the diameter and general geometry of the flow channel. Such precise knowledge is, however, seldom possible, especially under conditions where real-time monitoring is desired.

One blood characteristic that can be obtained with minimal or no invasion is blood pressure. In addition to causing minimal patient trauma, blood pressure measurement technology has the added benefit of being accurate.

Many blood pressure measurement systems rely on the pulse contour method (PCM), which calculates an estimate of one or more cardiac parameters of interest, such as CO, from characteristics of a blood pressure waveform. In the PCM, “Windkessel” parameters, such as characteristic impedance of the aorta, compliance, and total peripheral resistance, are often used to construct a linear or non-linear, hemodynamic model of the aorta. In essence, blood flow is analogized to a flow of electrical current in a circuit in which an impedance is in series with a parallel-connected resistance and capacitance (compliance). The three required parameters of the model are usually determined either empirically, through a complex calibration process, or from compiled “anthropometric” data, i.e., data about the age, sex, height, weight, and/or other parameters of other patients or test subjects. U.S. Pat. No. 5,400,793 (Wesseling, 28 Mar. 1995) and U.S. Pat. No. 5,535,753 (Petrucelli, et al., 16 Jul. 1996) disclose systems that rely on a Windkessel circuit model to determine CO.

PCM-based systems can monitor SV-derived cardiac parameters using blood pressure measurements taken using a variety of measurement apparatus, such as a finger cuff, and can do so more or less continuously. This ease of use comes at the potential cost of accuracy, however, as the PCM can be no more accurate than the rather simple, three-parameter model from which it was derived. A model of a much higher order would be needed to faithfully account for other phenomena. Many improvements, with varying degrees of complexity, have been proposed for improving the accuracy of the basic PCM model.

Recently, several studies have confirmed the clinical significance of monitoring the variations observed in left ventricular stroke volume that result from the interaction of the cardiovascular system and the lungs under mechanical ventilation. These stroke volume variations (SVV) are caused by the cyclic increases and decreases in the intrathoracic pressure due to the mechanical ventilation, which lead to variations in the cardiac preload and afterload. SVV has recently been extensively investigated and several studies have shown the usefulness of using SVV as a predictor of preload dependence and fluid responsiveness in various clinical situations. Several other parameters based on SVV have been found to be useful as well. In particular, systolic pressure variation (SPV) with its delta-Up (ΔUp) and delta-Down (ΔDown) components has been found to be a very useful predictor of preload dependence and fluid responsiveness. SPV is based on the changes in the arterial pulse pressure due to respiration-induced variations in stroke volume. Yet another parameter that has recently been investigated and shown to be a valid indicator of preload dependence and fluid responsiveness is the pulse pressure variation (PPV).

These recent developments in arterial pulse contour analysis methods have opened unique opportunities for less-invasive, continuous and real-time estimation of SVV. This allows clinicians to use SVV routinely along with SV and CO in their assessment of the hemodynamic state of critical care patients.

Existing systems for measuring preload dependence and fluid responsiveness based on respiration-induced changes in the arterial pulse pressure are almost all based on one of only a few methods. Some of the methods described in the literature include the measurements of Pulse Pressure Variation (PPV), Systolic Pressure Variation (SPV) and Stroke Volume Variation (SVV).

PPV estimation is based on Equation 1:

$$PPV = 100 \times \{ \frac{( {PP}_{max} - {PP}_{min} )}{[ \frac{1}{2} ( {PP}_{max} + {PP}_{min} ) ]} \} \qquad ( Equation 1 )$$

where PP is the measured pulse pressure, and PPₘₐₓ and PPₘᵢₙ are, respectively, the maximum and the minimum peak-to-peak values of the pulse pressure during one respiratory (inspiration-expiration) cycle.

SPV estimation is based on Equation 2:

$$SPV = 100 \times \{ \frac{( {SP}_{max} - {SP}_{min} )}{[ \frac{1}{2} ( {SP}_{max} + {SP}_{min} ) ]} \} \qquad ( Equation 2 )$$

where SP is the measured systolic pressure, and SPₘₐₓ and SPₘᵢₙ are, respectively, the maximum and minimum values of the systolic pressure during one respiratory cycle.

Similarly, SVV estimation is based on Equation 3:

$$SVV = 100 \times \{ \frac{( {SV}_{max} - {SV}_{min} )}{[ \frac{1}{2} ( {SV}_{max} + {SV}_{min} ) ]} \} \qquad ( Equation 3 )$$

where SV is the stroke volume, and SVₘₐₓ and SVₘᵢₙ are, respectively, the maximum and minimum values of the stroke volume during one respiratory cycle.

In Equations 1, 2, and 3, the denominators are the averages of the maximum and minimum values of PP, SP and SV, respectively. In other words, the denominators are mean values, albeit of only two measurement points. This simple averaging of extreme values has been most common merely to simplify the calculations, which have typically been performed by hand. More reliable values may be obtained, however, by using the mean of all the measurement values over the measurement interval, that is, the first statistical moment of PP, SP, and SV.

Thus, for each of PPV, SPV and SVV, the respective variation value formula expresses the magnitude of the range of the value (maximum minus minimum) relative to the mean of the extreme (maximum and minimum) values.

The specific monitoring of SVV has both specific difficulties and advantages. Physiologically, SVV is based on several complex mechanisms of cardio-respiratory interaction. In brief: mechanical ventilation causes changes in left ventricular preload, which leads to distinct variations in left ventricular stroke volume and systolic arterial pressure. Monitoring of SVV enables prediction of left ventricular response to volume administration and helps with correct assessment of hypovolemia and the subsequent decision to undertake volume resuscitation in many critical situations.

### SUMMARY

Methods for determining a cardiovascular parameter reflecting preload dependence fluid responsiveness or volume responsiveness are disclosed. These methods involve receiving a waveform dataset corresponding to an arterial blood pressure signal, or any signal proportional to, or derived from the arterial blood pressure signal, such as pulse oximetry (pulseox), Doppler ultrasound, or bioimpedance signal, and analyzing the signal to detect premature ventricular and/or atrial contractions. If any premature ventricular and/or atrial contractions are present, they are removed from the waveform dataset. Once the premature ventricular and/or atrial contractions are removed, a cardiovascular parameter reflecting preload dependence and fluid responsiveness or volume responsiveness using the modified waveform dataset can be calculated. Removal of the premature ventricular and/or atrial contraction data from the dataset increases the accuracy and sensitivity of calculations performed on the dataset waveform.

The methods for detecting a premature ventricular and/or atrial contraction disclosed herein include identifying an individual cardiac cycle in the waveform/signal dataset and comparing one or more parameters of the individual cardiac cycle to one or more parameters of a control cardiac cycle. As used herein, the term waveform dataset refers to a set of data corresponding to a signal, e.g., an arterial blood pressure signal or any signal proportional to, or derived from the arterial blood pressure signal, such as pulse oximetry (pulseox), Doppler ultrasound, or bioimpedance signal. The individual cardiac cycle is identified as a premature ventricular or atrial contraction if the one or more parameters of the individual cardiac cycle differs from the one or more parameters of the control cardiac cycle by a predetermined amount. Individual or multiple parameters of the cardiac cycle can be used for comparison.

Methods for detecting arrhythmia are also disclosed. These methods involve receiving a waveform dataset corresponding to an arterial blood pressure signal, or any signal proportional to or derived from the arterial blood pressure signal, such as pulseox, Doppler ultrasound or bioimpedance signal and analyzing the waveform to detect premature ventricular or atrial contractions. If the number of premature ventricular or atrial contractions exceeds a predetermined arrhythmia threshold, a user, such as a is medical professional, is notified. Also, if the variability of one or more parameters of the individual cardiac cycles, exceeds a predetermined threshold, the respective interval is considered an arrhythmia intervals and, a user, such as a medical professional, is notified. The methods for detecting premature ventricular or atrial contractions are the same as those described above.

### DESCRIPTION OF DRAWINGS

FIG. 1 is an atrial pressure versus time ( 1/100^{th} second increments) waveform displaying several cardiac cycles.

FIG. 2 is an atrial pressure versus time ( 1/100th second increments) waveform that contains two premature ventrical contractions.

FIG. 3 is an atrial pressure versus time ( 1/100th second increments) waveform showing three cardiac cycles.

FIG. 4 is an atrial pressure versus time ( 1/100th second increments) waveform annotated to indicate the duration of a cardiac cycle (t_{c}).

FIG. 5 is an atrial pressure versus time ( 1/100th second increments) waveform annotated to indicate the duration of a systole (tₛ) and the duration of a diastole (t_{d}).

FIG. 6 is an atrial pressure versus time ( 1/100th second increments) waveform annotated to indicate the duration of a systolic rise (tᵣ) and the duration of a systolic decay (t_{dec}).

FIG. 7 is an atrial pressure versus time ( 1/100th second increments) waveform annotated to indicate the duration of the overall decay (tₒᵥ_{_{—}}_{dec}).

Like reference numerals and symbols in the various drawings indicate like elements.

### DETAILED DESCRIPTION

Disclosed herein are methods for determining a cardiovascular parameter reflecting fluid or volume responsiveness by using a waveform dataset corresponding to a signal, for example, from an arterial blood pressure, or any signal proportional to, or derived from the arterial pressure signal such as pulseox signal, Doppler ultrasound or bioimpedance measurement device. These methods involve detecting premature ventricular or atrial contractions and removing these contractions from the waveform dataset prior to calculating the cardiovascular parameter. The premature ventricular or atrial contractions are detected by a variety of methods.

Also disclosed herein are methods of detecting arrhythmia by using a waveform dataset corresponding to a signal, for example, from an arterial blood pressure or any signal proportional to, or derived from the arterial pressure signal such as, pulseox, Doppler ultrasound or bioimpedance measurement device. These methods involve detecting premature ventricular or atrial contractions. In these methods, a user such as a medical professional is notified if the number of premature ventricular or atrial contractions exceeds a predetermined arrhythmia threshold. The premature ventrical or atrial contractions are detected by a variety of methods.

Determining a cardiovascular parameter reflecting preload dependence, fluid responsiveness, or volume responsiveness according to the methods described herein involves receiving a waveform or a signal dataset. As used herein, the term waveform dataset refers to a set of data corresponding to a signal, e.g., an arterial blood pressure signal, or any signal proportional to, or derived from the arterial blood pressure signal, such as pulse oximetry (pulseox), Doppler ultrasound, or bioimpedance signal. This dataset is then analyzed to detect any premature ventricular or atrial contractions. If any premature ventricular or atrial contractions are detected, these premature ventrical or atrial contractions are removed from the waveform dataset. The resulting waveform dataset is referred to herein as a modified waveform dataset. Finally, a cardiovascular parameter reflecting preload dependence) fluid responsiveness, or volume responsiveness is calculated using the modified waveform dataset.

Detecting premature ventricular or atrial contractions can be accomplished by identifying an individual cardiac cycle in a waveform dataset and comparing one or more parameters of the individual cardiac cycle to one or more parameters of a control cardiac cycle. Premature ventricular or atrial contractions are identified by comparing the one or more parameters of an individual cardiac cycle with the same one or more parameters from a control cardiac cycle. If the one or more parameters of the individual cardiac cycle differ by a predetermined threshold amount from the same one or more parameters from the control cardiac cycle, the individual cardiac cycle is identified as a premature ventricular or atrial contraction.

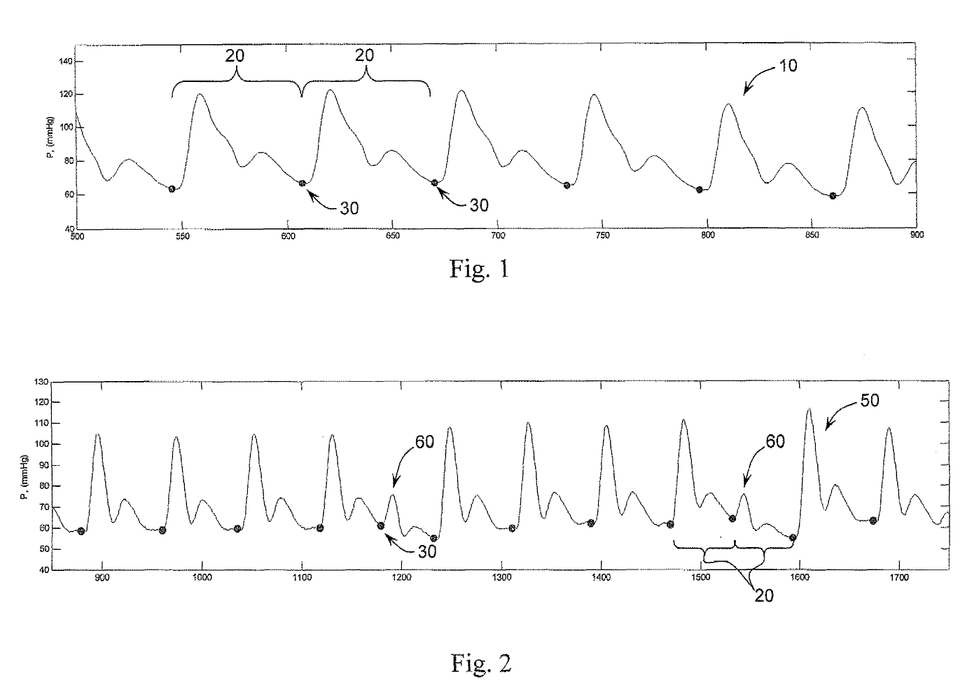

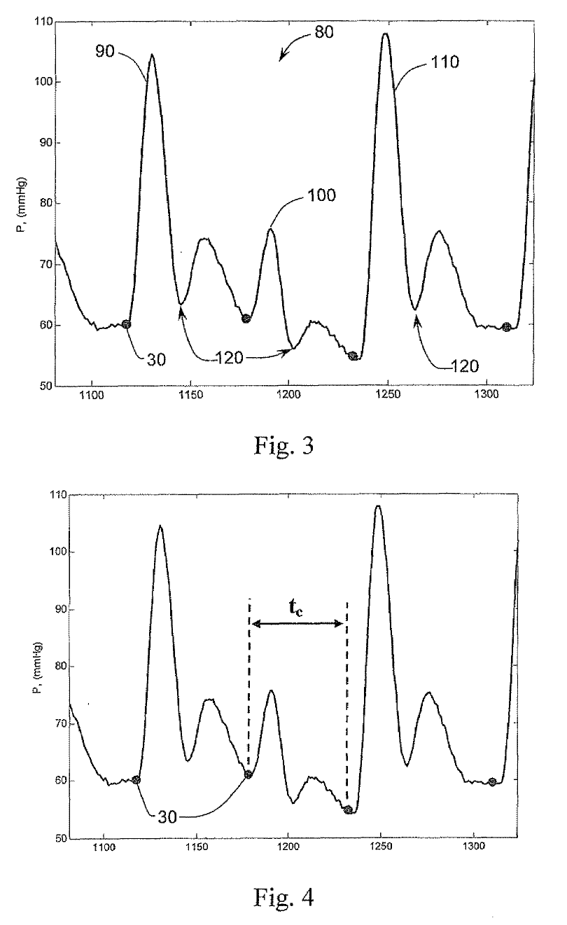

The parameters used for comparison are statistical and other measurements based on portions or phases of a cardiac cycle. The portions of a cardiac cycle used herein by way of example are shown in FIGS. 1-7. In each of FIGS. 1-7, the x-axis units are 100ths of a second (e.g., 100 x-axis units corresponds to 1 second and 200 x-axis units corresponds to 2 seconds). FIG. 1 shows an atrial pressure waveform 10 with several cardiac cycles 20. The dots along the atrial pressure waveform 10 indicate the end-diastolic pressure 30 of one cardiac cycle and the start of the next cardiac cycle. FIG. 2 shows an atrial pressure waveform 50 with two premature ventrical contractions 60. The premature ventrical contractions 60 in FIG. 2 generated cardiac cycles with less pressure when compared to the other cardiac cycles 20. FIG. 3 shows an atrial pressure waveform 80 with three cardiac cycles (90, 100, and 110). The middle cardiac cycle 100 represents a premature ventricular contraction. The inflection point of an arterial pressure waveform of a cardiac cycle that defines the end of the systolic phase and the beginning of the diastolic phase is called a dichroic notch 120.

The ending/starting point of a cardiac cycle 30 and the dichroic notch 120 provide starting and ending points for defining various parameters used with the methods described herein. The parameters used herein include the entire cardiac cycle, the systole, the diastole, the systolic rise, the systolic decay, and the overall decay of an arterial pressure signal. The time components of each of these parameters are also used, i.e., useful parameters include duration of the entire cardiac cycle (t_{c}), duration of the systole (tₛ), duration of the diastole (t_{d}), duration of the systolic rise (tᵣ), duration of the systolic decay (t_{dec}), and duration of the overall decay (tₒᵥ_{_{—}}_{dec}).

The duration of a cardiac cycle, t_{c}, is shown in FIG. 4. As shown, t_{c} is the time between the start point 30 of the cardiac cycle and the end point of the cardiac cycle.

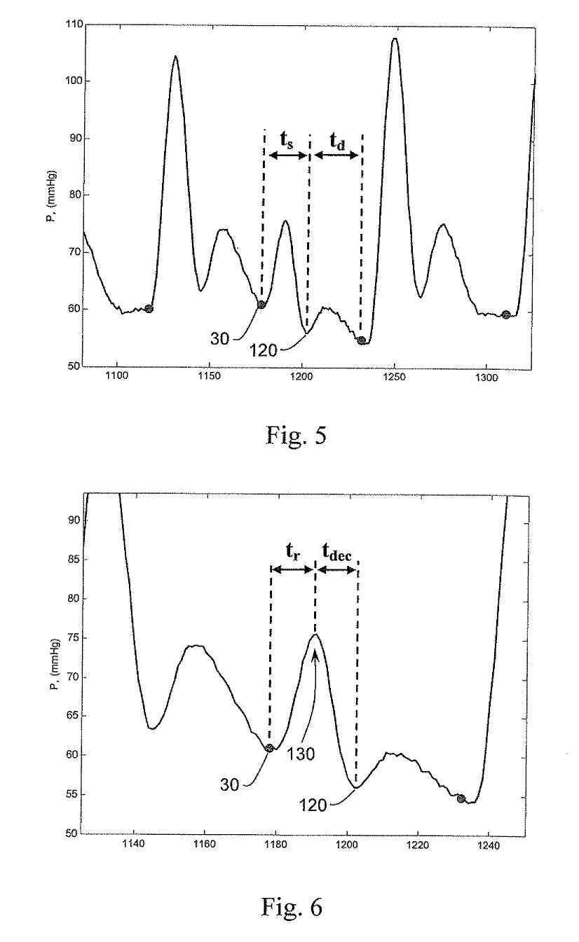

The duration of a systole, tₛ, is shown in FIG. 5. As shown, tₛ is the time between the start point 30 of the cardiac cycle and the dichroic notch 120 of the cardiac cycle.

The duration of the diastole, t_{d}, is also shown in FIG. 5. As shown, t_{d} is the time between the dichroic notch 120 of the cardiac cycle and the end point of the cardiac cycle.

The duration of a systolic rise, tᵣ, is shown in FIG. 6. As shown, tᵣ is the time from the start point 30 of the cardiac cycle to the maximum point 130 of the initial increase in arterial pressure after the onset of the systole.

The duration of the systolic decay, t_{dec}, is also shown in FIG. 6. As shown, t_{dec} is the time from the maximum point 130 of the initial increase in arterial pressure after the onset of the systole to the dichroic notch 120.

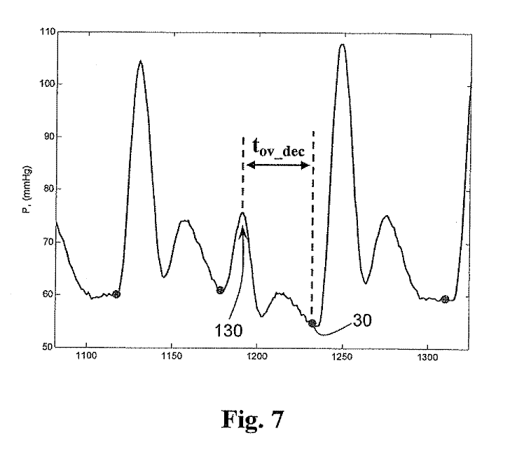

The duration of the overall decay, tₒᵥ_{_{—}}_{dec}, is shown in FIG. 7. As shown, tₒᵥ_{_{—}}_{dec} is the time from the maximum point 130 of the initial increase in arterial pressure after the onset of the systole to the end point of the cardiac cycle.

One method to detect a premature ventricular or atrial contraction is to analyze the durations of the different phases of the cardiac cycle, i.e., time intervals of the different phases, of an arterial waveform/signal as just described are compared. The methods described herein, for example, compare the duration of the entire cardiac cycle (i.e. the beat heart rate), the duration of the systole, the duration of the diastole, the duration of the systolic rise, the duration of the systolic decay, and/or the duration of the entire decay.

Another method to detect a premature ventricular or atrial contraction is to analyze the location of the dichroic notches of an arterial waveform/signal. For example, the location of a dichroic notch versus the maximum systolic pressure and the location of a dichroic notch versus the diastolic pressure (the minimum pressure of the cardiac cycle before the maximum systolic pressure) are analyzed.

To detect a premature ventrical or atrial contraction, the statistical characteristics, i.e., statistical moments, of the different portions of an arterial waveform as just described are compared. In the methods described herein the first four statistical moments, i.e., mean, variance, skewness, and kurtosis, are used. The following equations can be used to calculate the first four statistical moments (where N is the total number of samples during systole):

$$\begin{matrix}Mean \text{:} & \\ {μ}_{1 p} = \frac{1}{N - 1} {\sum }_{k = 0}^{N - 1} P ( k ) & ( Equation 4 ) \\ Variance \text{:} & \\ {μ}_{2 p} = {σ}_{o}^{2} = \frac{1}{N - 1} {\sum }_{k = 0}^{N - 1} {( P ( k ) - {P}_{avg} )}^{2} & ( Equation 5 ) \\ Skewness \text{:} & \\ {μ}_{3 p} = \frac{1}{N - 1} {\sum }_{k = 0}^{N - 1} {( \frac{( P ( k ) - {P}_{avg}}{{σ}_{p}} )}^{3} & ( Equation 6 ) \\ Kurtosis \text{:} & \\ {μ}_{4 p} = \frac{1}{N - 1} {\sum }_{k = 0}^{N - 1} {( \frac{( P ( k ) - {P}_{avg}}{{σ}_{p}} )}^{4} & ( Equation 7 )\end{matrix}$$

Additional characteristics that can be used to compare cardiac cycles include the power of the phases of the cardiac cycles as discussed above as well as frequency characteristics and time-frequency characteristics of the phases. The power of a phase of the cardiac cycle is measured as the integral of the cardiac signal under each phase. The power can be calculated by integrating the signal within each phase. Thus, for example, the power of the systole phase, E_{sys}, can be calculated using the following equation (where N is the total number of samples during systole):

$${E}_{sys} = {\sum }_{k = 0}^{k = N - 1} P ( k ) \qquad ( Equation 8 )$$

The frequency characteristics of each phase of a cardiac cycle can be derived by performing a Fourier transform analysis. Various known Fourier transforms including fast Fourier transforms can be used.

The time-frequency characteristics of each phase of a cardiac cycle can be derived using wavelet transform analysis. Wavelet analysis is well suited for analyzing signals which have transients or other non-stationary characteristics in the time domain. In contrast to Fourier transforms, wavelet analysis retains information in the time domain, i.e., when the event occurred.

In comparing statistical or other characteristics or parameters of one or more portions of a cardiac cycle to a control cardiac cycle, different approaches can be used. For example, one or more characteristics of a cardiac cycle can be compared to the same characteristic(s) of the cardiac cycle immediately preceding the cardiac cycle being examined, i.e., the control cardiac cycle is the cardiac cycle immediately preceding the cardiac cycle being examined. Another comparison can involve comparing one or more characteristics of a cardiac cycle with the same characteristic(s) of the cardiac cycle immediately following the cardiac cycle being examined, i.e., the control cardiac cycle is the cardiac cycle immediately following the cardiac cycle being examined. A further comparison can involve comparing one or more characteristics of a cardiac cycle with both the cardiac cycle immediately preceding the cardiac cycle being examined and the cardiac cycle immediately following the cardiac cycle being examined, i.e. the control cardiac cycles are the cardiac cycle immediately preceding the cardiac cycle being examined and the cardiac cycle immediately following the cardiac cycle being examined. An additional comparison can involve comparing one or more characteristics of a cardiac cycle with the same characteristic(s) in a median cardiac cycle from a sequence containing at least three cardiac cycles, i.e., the control cardiac cycle is a median cardiac cycle from a sequence containing at least three cardiac cycles. Another comparison can involve comparing one or more characteristics of a cardiac cycle with the same characteristic(s) in a statistical measurement of a phase of a cardiac cycle, i.e., the control cardiac cycle is a statistical representation of the measurement being compared. These comparison examples have been presented as comparisons of one or more characteristics, however, as will be apparent to one of skill in the art, multiple parameters for individual or multiple portions of the cardiac cycle can be used. Further, as will also be apparent to one of skill in the art, as these methods are likely to be performed using computer devices, a large number of these comparisons can be performed in real time.

In making such comparisons, predetermined thresholds can be used. As used herein, a predetermined threshold is a value assigned prior to a comparison being made. Generally, the predetermined threshold for a parameter will indicate a value related to a control cardiac cycle as measured, for example, from the subject being monitored, from averaged, or from anthropomorphic data. Depending on the parameter measured, the predetermined threshold can be a very small value or difference, or could be a larger value. Such predetermined thresholds will be easily provided by a medical professional or instrument operator. The predetermined threshold amount selected for a particular parameter will depend on the accuracy of the particular parameter used.

For example, if a single parameter is used, a predetermined threshold amount can be a difference of 30 percent or more as compared to the same parameter of the control cardiac cycle, a difference of 25 percent or more as compared to the same parameter of the control cardiac cycle, a difference of 20 percent or more as compared to the same parameter of the control cardiac cycle, a difference of 15 percent or more as compared to the same parameter of the control cardiac cycle, a difference of 10 percent or more as compared to the same parameter of the control cardiac cycle, a difference of 5 percent or more as compared to the same parameter of the control cardiac cycle, a difference of 4 percent or more as compared to the same parameter of the control cardiac cycle, a difference of 3 percent or more as compared to the same parameter of the control cardiac cycle, a difference of 2 percent or more as compared to the same parameter of the control cardiac cycle, a difference of 1 percent or more as compared to the same parameter of the control cardiac cycle, a difference of 0.5 percent or more as compared to the same parameter of the control cardiac cycle, a difference of 0.4 percent or more as compared to the same parameter of the control cardiac cycle, a difference of 0.3 percent or more as compared to the same parameter of the control cardiac cycle, a difference of 0.2 percent or more as compared to the same parameter of the control cardiac cycle, or a difference of 0.1 percent or more as compared to the same parameter of the control cardiac cycle.

Further, if more than one parameter is used, the predetermined threshold amount will depend on the particular combination of parameters used in combination with the accuracy of the parameter measurements. For example, if more than one parameter is used, a predetermined threshold amount can be a difference of 30 percent or more as compared to the same one or more parameters of the control cardiac cycle, a difference of 25 percent or more as compared to the same one or more parameters of the control cardiac cycle, a difference of 20 percent or more as compared to the same one or more parameters of the control cardiac cycle, a difference of 15 percent or more as compared to the same one or more parameters of the control cardiac cycle, a difference of 10 percent or more as compared to the same one or more parameters of the control cardiac cycle, a difference of 5 percent or more as compared to the same one or more parameters r of the control cardiac cycle, a difference of 4 percent or more as compared to the same one or more parameters of the control cardiac cycle, a difference of 3 percent or more as compared to the same one or more parameters of the control cardiac cycle, a difference of 2 percent or more as compared to the same one or more parameters of the control cardiac cycle, a difference of 1 percent or more as compared to the same one or more parameters of the control cardiac cycle, a difference of 0.5 percent or more as compared to the same one or more parameters of the control cardiac cycle, a difference of 0.4 percent or more as compared to the same parameter of the control cardiac cycle, a difference of 0.3 percent or more as compared to the same parameter of the control cardiac cycle, a difference of 0.2 percent or more as compared to the same parameter of the control cardiac cycle, or a difference of 0.1 percent or more as compared to the same parameter of the control cardiac cycle. Typically, the greater the number of parameters used, the lower the predetermined threshold amounts are for each parameter.

In addition to the predetermined thresholds, all the parameters used for an analysis can be assembled in a single parameters data set. In a dataset, the accuracy of a particular parameter defines the weight of the parameter in the parameters data set. Based on the weight of a respective parameter in the parameters dataset a threshold is assigned to each parameter and the number of parameters from the parameters dataset exceeding the predetermined thresholds are counted. When multiple parameters are used, each parameter can have its own predetermined threshold amount. For example, a predetermined threshold amount can be a difference of 30 percent or more as compared to the same parameter of the control cardiac cycle, a difference of 25 percent or more as compared to the same parameter of the control cardiac cycle, a difference of 20 percent or more as compared to the same parameter of the control cardiac cycle, a difference of 15 percent or more as compared to the same parameter of the control cardiac cycle, a difference of 10 percent or more as compared to the same parameter of the control cardiac cycle, a difference of 5 percent or more as compared to the same parameter of the control cardiac cycle, a difference of 4 percent or more as compared to the same parameter of the control cardiac cycle, a difference of 3 percent or more as compared to the same parameter of the control cardiac cycle, a difference of 2 percent or more as compared to the same parameter of the control cardiac cycle, a difference of 1 percent or more as compared to the same parameter of the control cardiac cycle, or a difference of 0.5 percent or more as compared to the same parameter of the control cardiac cycle. As a specific example, a first parameter could have a predetermined threshold amount of a difference of 15 percent or more as compared to the same parameter of the control cardiac cycle and a second parameter could have a predetermined threshold amount of a difference of 4 percent or more as compared to the same parameter of the control cardiac cycle. The number of predetermined threshold amounts can be equal to or less than the number of parameters evaluated.

Once a premature ventricular or atrial contraction is detected the signal is removed from the waveform dataset. For example, in the waveform dataset provided in FIG. 3, cardiac cycle 100 representing a premature ventricular contraction would be removed from the waveform dataset and the calculations would be based just on the preceding and following cardiac cycles 90 and 110. Removal of the premature ventricular or atrial contraction data from the waveform dataset increases the accuracy and sensitivity of calculations performed on the dataset. Therefore, calculations such as left ventricular stroke volume variation, pulse pressure variation, or systolic pressure variation achieve increased accuracy and sensitivity when premature ventricular or atrial contraction data is removed. An example of a ventricular stroke volume variation calculation is provided in U.S. Patent Application Publication No. US 2005/0187481, which is incorporated by reference herein in its entirety.

To achieve even greater sensitivity and accuracy, the methods described above can include the additional step of removing the signal for the cardiac cycle immediately following the premature ventricular or atrial contraction from the waveform dataset (e.g. cardiac cycle 110 from FIG. 3). This additional subtraction can be performed as a precaution because the cardiac cycle that follows a premature ventricular or atrial contraction can generate higher pressure than the rest of the normal cardiac cycles and could, therefore, affect the calculation of a cardiovascular parameter reflecting fluid or volume changes.

In addition to the removal of premature ventricular or atrial contractions, other operations can be performed on the dataset to increase the accuracy and sensitivity of calculations performed on the waveform dataset. For example, the signal can be filtered to reduce the effect of noise, interference) and artifacts that may occur in the signal. Such filtering can be accomplished through the use of a low-pass filter for example. Following filtering, large motion artifacts can be detected and removed from the waveform dataset. Such artifacts are common as they often result from patient movement or from flushing of an arterial line. Additionally, bad cardiac cycles can be removed after beat detection before detecting premature ventrical or atrial contractions.

Once identified, a premature ventricular or atrial contraction can be indicated on a graphical user interface. When the waveform dataset corresponding to an arterial blood pressure, or any signal proportional to or derived from the arterial pressure signal, such as pulseox, Doppler ultrasound, or bioimpedance signal is displayed on a graphical user interface simultaneously with the detection step of the methods described herein, indications that premature ventricular or atrial contractions are present generally or a specific indication that a particular cardiac cycle is a premature ventricular or atrial contraction can be provided. The same information can be provided for data not shown in real time.

The time period for the waveform dataset can be a set value, for example, the time period can be about ten minutes or more, about five minutes of more, about four minutes or more, about three minutes or more, about two minutes or more, about one minute or more, about 50 seconds or more, about 40 seconds or more, about 30 seconds or more, about 20 seconds or more, or about 10 seconds or more. For example, the time period can be about ten, about nine, about eight, about seven, about six, about five, about four, about three, about two, or about one minutes. Further, for example, the time period can be about 55, about 50, about 45, about 40, about 35, about 30, about 25, about 20, about 15, about 10, or about 5 seconds. This time period can be constant or can be increased. Further, if premature ventricular or atrial contractions are detected, the time period for the waveform dataset can be increased. Such an increase in sample time may improve detection ability and the consistency of the data.

Also disclosed herein is a method of detecting arrhythmia. This method of detecting arrhythmia involves receiving a waveform dataset. The waveform dataset can correspond to a signal, for example, from an arterial blood pressure, or any signal proportional to or derived from the arterial pressure signal, such as pulseox, Doppler ultrasound or bioimpedance measurement device. This dataset is then analyzed to detect any premature ventricular or atrial contractions. If the premature ventricular or atrial contractions exceed a predetermined arrhythmia threshold, a user such as a medical professional is notified. If the predetermined arrhythmia threshold is met, the data indicates that the patient being monitored has arrhythmic cardiac cycles in excess of the arrhythmia threshold.

The arrhythmia threshold can be based on a percentage of premature ventricular or atrial contractions as calculated based on the total number of cardiac cycles measured. For example, the predetermined arrhythmia threshold can be about 30% of the total number of cardiac cycles measured, about 25% of the total number of cardiac cycles measured, about 20% of the total number of cardiac cycles measured, about 15% of the total number of cardiac cycles measured, or about 10% of the total number of cardiac cycles measured. The predetermined arrhythmia threshold can be established by one of skill in the art based on the percentage of premature ventricular or atrial contractions that will aid in monitoring a patient. The total number of cardiac cycles measured can also be established by one of skill in the art.

Detecting premature ventricular or atrial contractions in this method of detecting arrhythmia can be accomplished using the same methods, characteristics, and parameters described above. Additionally, arrhythmia detection can be accomplished by detecting variability in the time, statistical or energy/power parameter of the arterial pressure signal, or any signal proportional to or derived from the arterial pressure signal. If the variability of a selected parameter or parameters exceeds a predetermined variability as compared to a control cardiac cycle, the cycle to which the parameter is related is identified as a premature ventricular or atrial contraction. The waveform dataset can be processed in the same way as discussed above.

If a single parameter is used, for example, a predetermined variability can be 30 percent or more as compared to the same parameter of the control cardiac cycle, a variability of 25 percent or more as compared to the same parameter of the control cardiac cycle, a variability of 20 percent or more as compared to the same parameter of the control cardiac cycle, a variability of 15 percent or more as compared to the same parameter of the control cardiac cycle, a variability of 10 percent or more as compared to the same parameter of the control cardiac cycle, a variability of 5 percent or more as compared to the same parameter of the control cardiac cycle, a variability of 4 percent or more as compared to the same parameter of the control cardiac cycle, a variability of 3 percent or more as compared to the same parameter of the control cardiac cycle, a variability of 2 percent or more as compared to the same parameter of the control cardiac cycle, a variability of 1 percent or more as compared to the same parameter of the control cardiac cycle, a difference of 0.5 percent or more as compared to the same parameter of the control cardiac cycle, a difference of 0.4 percent or more as compared to the same parameter of the control cardiac cycle, a difference of 0.3 percent or more as compared to the same parameter of the control cardiac cycle, a difference of 0.2 percent or more as compared to the same parameter of the control cardiac cycle, or a difference of 0.1 percent or more as compared to the same parameter of the control cardiac cycle.

Further, if more than one parameter is used, the predetermined variability will depend on the particular combination of parameters used in combination with the accuracy of the parameter measurements. For example, if more than one parameter is used, a predetermined variability can be 30 percent or more as compared to the same one or more parameters of the control cardiac cycle, a variability of 25 percent or more as compared to the same one or more parameters of the control cardiac cycle, a variability of 20 percent or more as compared to the same one or more parameters of the control cardiac cycle, a variability of 15 percent or more as compared to the same one or more parameters of the control cardiac cycle, a variability of 10 percent or more as compared to the same one or more parameters of the control cardiac cycle, a variability of 5 percent or more as compared to the same one or more parameters of the control cardiac cycle, a variability of 4 percent or more as compared to the same one or more parameters of the control cardiac cycle, a variability of 3 percent or more as compared to the same one or more parameters of the control cardiac cycle, a variability of 2 percent or more as compared to the same one or more parameters of the control cardiac cycle, a variability of 1 percent or more as compared to the same one or more parameters of the control cardiac cycle, a difference of 0.5 percent or more as compared to the same parameter of the control cardiac cycle, a difference of 0.4 percent or more as compared to the same parameter of the control cardiac cycle, a difference of 0.3 percent or more as compared to the same parameter of the control cardiac cycle, a difference of 0.2 percent or more as compared to the same parameter of the control cardiac cycle, or a difference of 0.1 percent or more as compared to the same parameter of the control cardiac cycle. Typically, the greater the number of parameters used, the lower the predetermined variability amounts are for each parameter.

When multiple parameters are used, each parameter can have its own predetermined variability. For example, a predetermined variability can be 30 percent or more as compared to the same parameter of the control cardiac cycle, a variability of 25 percent or more as compared to the same parameter of the control cardiac cycle, a variability of 20 percent or more as compared to the same parameter of the control cardiac cycle, a variability of 15 percent or more as compared to the same parameter of the control cardiac cycle, a variability of 10 percent or more as compared to the same parameter of the control cardiac cycle, a variability of 5 percent or more as compared to the same parameter of the control cardiac cycle, a variability of 4 percent or more as compared to the same parameter of the control cardiac cycle, a variability of 3 percent or more as compared to the same parameter of the control cardiac cycle, a variability of 2 percent or more as compared to the same parameter of the control cardiac cycle, a variability of 1 percent or more as compared to the same parameter of the control cardiac cycle, a difference of 0.5 percent or more as compared to the same parameter of the control cardiac cycle, a difference of 0.4 percent or more as compared to the same parameter of the control cardiac cycle, a difference of 0.3 percent or more as compared to the same parameter of the control cardiac cycle, a difference of 0.2 percent or more as compared to the same parameter of the control cardiac cycle, or a difference of 0.1 percent or more as compared to the same parameter of the control cardiac cycle. As a specific example, a first parameter could have a predetermined variability of 15 percent or more as compared to the same parameter of the control cardiac cycle and a second parameter could have a predetermined variability of 4 percent or more as compared to the same parameter of the control cardiac cycle. The number of predetermined variabilities can be equal to or less than the number of parameters evaluated.

Once arrhythmia has been identified using this method, a user such as medical professional can be notified that arrhythmia has been detected by conventional methods, such as by a sound or an indication on a graphical user interface. For example, when patient data is displayed on a graphical user interface, the graphical user interface can also indicate that arrhythmia has been detected.

As used herein the term “arterial blood pressure” refers to the force exerted by circulating blood on the walls of blood vessels and an “arterial blood pressure signal” is a signal from a blood pressure monitoring instrument such as a sphygmomanometer or other pressure transducer. As used herein the term “pulseox” refers to a signal from a pulse oximeter, which is an instrument that indirectly measures the amount of oxygen in a subject's blood using various characteristics of light absorption. As used herein the term “bioimpedance signal” refers to a signal from a bioimpedance plethysmography device, i.e., a device that measures blood parameters such as pulsatile blood volume changes in the aorta. As used herein, the term “Doppler ultrasound” refers to a signal from a Doppler ultrasound device, a device that makes Doppler enhanced ultrasound measurements.

The methods described herein can be implemented by a computer program loadable onto a computer unit or a processing system in order to execute the described methods. Moreover, the methods can be stored as computer-executable instructions on a computer readable medium to allow the methods to be loaded into and executed by different operating systems.

The methods disclosed herein are equally applicable to any subject for which an arterial blood pressure, pulseox, Doppler ultrasound, or bioimpedance signal can be detected. For example, the subject can be, but is not limited to a mammal such as a human.

The present invention is not limited in scope by the embodiments disclosed herein which are intended as illustrations of a few aspects of the invention and any embodiments which are functionally equivalent are within the scope of this invention. Various modifications of the methods in addition to those shown and described herein will become apparent to those skilled in the art and are intended to fall within the scope of the appended claims. Further, while only certain representative combinations of the method steps disclosed herein are specifically discussed in the embodiments above, other combinations of the method steps will become apparent to those skilled in the art and also are intended to fall within the scope of the appended claims. Thus a combination of steps may be explicitly mentioned herein; however, other combinations of steps are included, even though not explicitly stated. The term “comprising” and variations thereof as used herein is used synonymously with the term “including” and variations thereof and are open, non-limiting terms.

What is claimed is:

1. A method of determining a cardiovascular parameter reflecting preload dependence, fluid responsiveness or, volume responsiveness comprising:
receiving a waveform dataset corresponding to an arterial blood pressure, or a signal proportional to, or derived from, the arterial blood pressure signal;
detecting a premature ventricular or atrial contraction;
removing the premature ventricular or atrial contraction from the waveform dataset to form a modified waveform dataset; and
calculating a cardiovascular parameter reflecting preload dependence, fluid responsiveness, or volume responsiveness using the modified waveform dataset.

2. The method of claim 1, wherein detecting a premature ventricular or atrial contraction comprises:
identifying an individual cardiac cycle in the waveform dataset;
comparing one or more parameters of the individual cardiac cycle to one or more parameters of a control cardiac cycle; and
identifying the individual cardiac cycle as a premature ventricular or atrial contraction if the one or more parameters of the individual cardiac cycle differs from the one or more parameters of the control cardiac cycle by a predetermined threshold amount.

3. The method of claim 2, wherein the predetermined threshold amount is 30% or more.

4. The method of claim 2, wherein the predetermined threshold amount is 25% or more.

5. The method of claim 2, wherein the predetermined threshold amount is 20% or more.

6. The method of claim 2, wherein the predetermined threshold amount is 15% or more.

7. The method of claim 2, wherein the predetermined threshold amount is 10% or more.

8. The method of claim 2, wherein the predetermined threshold amount is 5% or more.

9. The method of claim 2, wherein the predetermined threshold amount is 1% or more.

10. The method of claim 2, wherein the control cardiac cycle is a cardiac cycle immediately preceding the individual cardiac cycle.

11. The method of claim 10, further comprising comparing the individual cardiac cycle to the cardiac cycle immediately following the individual cardiac cycle.

12. The method of claim 2, wherein the control cardiac cycle is a cardiac cycle immediately following the individual cardiac cycle.

13. The method of claim 2, wherein the control cardiac cycle is a median cardiac cycle from a sequence containing at least three cardiac cycles.

14. The method of claim 2, wherein the control cardiac cycle is a mean cardiac cycle from a sequence containing at least three cardiac cycles.

15. The method of claim 2, wherein the one or more parameters is a statistical measurement of a phase of a cardiac cycle.

16. The method of claim 15, wherein the statistical measurement is one of average, variance, skewness, or kurtosis.

17. The method of claim 15, wherein the phase of a cardiac cycle is one of the entire cardiac cycle, systole, diastole, systolic rise, systolic decay, or overall decay.

18. The method of claim 17, wherein the one or more parameters is a time interval of the phase of a cardiac cycle.

19. The method of claim 18, wherein the time interval is measured from the end-diastolic pressure from the previous cardiac cycle.

20. The method of claim 2, wherein the one or more parameters is the power of a phase of a cardiac cycle.

21. The method of claim 20, wherein the phase of a cardiac cycle is selected from the group consisting of the entire cardiac cycle, systole, diastole, systolic rise, systolic decay, and overall decay.

22. The method of claim 2, wherein the one or more parameters is one or more frequency characteristics of a phase of a cardiac cycle.

23. The method of claim 22, wherein the phase of a cardiac cycle is selected from the group consisting of the entire cardiac cycle, systole, diastole, systolic rise, systolic decay, and overall decay.

24. The method of claim 2, wherein the one or more parameters is one or more time-frequency characteristics of a phase of a cardiac cycle.

25. The method of claim 24, wherein the phase of a cardiac cycle is selected from the group consisting of the entire cardiac cycle, systole, diastole, systolic rise, systolic decay, and overall decay.

26. The method of claim 1, wherein the cardiovascular parameter is left ventricular stroke volume variation, pulse pressure variation, or systolic pressure variation.

27. The method of claim 1, further comprising filtering the waveform dataset with a low pass filter.

28. The method of claim 1, further comprising subtracting the cardiac cycle following the premature ventricular or atrial contraction from the waveform dataset.

29. The method of claim 1, further comprising indicating the position of premature ventricular or atrial contractions on a graphical user interface.

30. The method of claim 1, further comprising when premature ventricular or atrial contraction are detected indicating that premature ventricular or atrial contractions are present on a graphical user interface.

31. The method of claim 1, wherein the waveform dataset is from a sampling period of a set duration.

32. The method of claim 31, wherein when premature ventricular or atrial contractions are detected, the duration of the sampling period is increased.

33. The method of claim 1, wherein the signal proportional to, or derived from, the arterial blood pressure signal is a pulseox, Doppler ultrasound, or bioimpedance signal.

34. A method of detecting arrhythmia comprising:
receiving a waveform dataset corresponding to an arterial blood pressure, or a signal proportional to, or derived from the arterial blood pressure signal;
detecting premature ventricular or atrial contractions; and
notifying a user if the number of premature ventrical or atrial contractions exceeds a predetermined arrhythmia threshold.

35. The method of claim 34, wherein the predetermined arrhythmia threshold is 30% of a total number of cardiac cycles.

36. The method of claim 34, wherein the predetermined arrhythmia threshold is 20% of a total number of cardiac cycles.

37. The method of claim 34, wherein the predetermined arrhythmia threshold is 15% of a total number of cardiac cycles.

38. The method of claim 34, wherein the predetermined arrhythmia threshold is 10% of a total number of cardiac cycles.

39. The method of claim 34, wherein detecting a premature ventrical or atrial contraction comprises:
identifying an individual cardiac cycle in the waveform dataset;
comparing one or more parameters of the individual cardiac cycle to one or more parameters of a control cardiac cycle; and
identifying the individual cardiac cycle as a premature ventricular or atrial contraction if the one or more parameters of the individual cardiac cycle differs from the one or more parameters of the control cardiac cycle by a selected parameter threshold.

40. The method of claim 39, wherein the selected parameter threshold difference is 30% or more.

41. The method of claim 39, wherein the selected parameter threshold difference is 25% or more.

42. The method of claim 39, wherein the selected parameter threshold difference is 20% or more.

43. The method of claim 39, wherein the selected parameter threshold difference is 15% or more.

44. The method of claim 39, wherein the selected parameter threshold difference is 10% or more.

45. The method of claim 39, wherein the selected parameter threshold difference is 5% or more.

46. The method of claim 39, wherein the predetermined threshold amount is 1% or more.

47. The method of claim 34, wherein detecting a premature ventrical or atrial contraction comprises:
identifying an individual cardiac cycle in the waveform dataset;
detecting variability in one or more parameters of the individual cardiac cycle as compared to a control cardiac cycle; and
identifying the individual cardiac cycle as a premature ventricular or atrial contraction if a predetermined variability in the one or more parameters of the individual cardiac cycle is met.

48. The method of claim 47, wherein the predetermined variability is 30% or more.

49. The method of claim 47, wherein the predetermined variability is 25% or more.

50. The method of claim 47, wherein the predetermined variability is 20% or more.

51. The method of claim 47, wherein the predetermined variability is 15% or more.

52. The method of claim 47, wherein the predetermined variability is 10% or more.

53. The method of claim 47, wherein the predetermined variability is 5% or more.

54. The method of claim 47, wherein the predetermined variability is 1% or more.

55. The method of claim 34, wherein the waveform dataset is from a sampling period of a set duration.

56. The method of claim 55, wherein when premature ventricular or atrial contractions are detected, the duration of the sampling period is increased.

57. The method of claim 39, wherein the control cardiac cycle is a cardiac cycle immediately preceding the individual cardiac cycle.

58. The method of claim 39, further comprising comparing the individual cardiac cycle to the cardiac cycle immediately after the individual cardiac cycle.

59. The method of claim 39, wherein the control cardiac cycle is a cardiac cycle immediately after the individual cardiac cycle.

60. The method of claim 39, wherein the control cardiac cycle is a median cardiac cycle from a sequence containing at least three cardiac cycles.

61. The method of claim 39, wherein the control cardiac cycle is a mean cardiac cycle from a sequence containing at least three cardiac cycles.

62. The method of claim 39, wherein the one or more parameters is a statistical measurement of a phase of a cardiac cycle.

63. The method of claim 62, wherein the statistical measurement is one of average, variance, skewness, or kurtosis.

64. The method of claim 62, wherein the phase of a cardiac cycle is one of the entire cardiac cycle, systole, diastole, systolic rise, systolic decay, or overall decay.

65. The method of claim 64, wherein the one or more parameters is a time interval of the phase of a cardiac cycle.

66. The method of claim 39, wherein the one or more parameters is the power of a phase of a cardiac cycle.

67. The method of claim 66, wherein the phase of a cardiac cycle is selected from the group consisting of the entire cardiac cycle, systole, diastole, systolic rise, systolic decay, and overall decay.

68. The method of claim 39, wherein the one or more parameters is one or more frequency characteristics of a phase of a cardiac cycle.

69. The method of claim 68, wherein the phase of a cardiac cycle is selected from the group consisting of the entire cardiac cycle, systole, diastole, systolic rise, systolic decay, and overall decay.

70. The method of claim 39, wherein the one or more parameters is one or more time-frequency characteristics of a phase of a cardiac cycle.

71. The method of claim 70, wherein the phase of a cardiac cycle is selected from the group consisting of the entire cardiac cycle, systole, diastole, systolic rise, systolic decay, and overall decay.

72. The method of claim 34, further comprising filtering the waveform dataset with a low pass filter.

73. The method of claim 34, wherein notifying a user comprises indicating arrhythmia on a graphical user interface.

74. The method of claim 34, wherein the signal proportional to, or derived from, the arterial blood pressure signal is a pulseox, Doppler ultrasound, or bioimpedance signal.
